(19) United States
(12) Patent Application Publication
(10) Pub. No.: US 2026/0148812 A1
(43) Pub. Date: May 28, 2026

(54) APPARATUS AND METHOD FOR MATCHING CLINICAL TRIAL CONTRACT RESEARCH ORGANIZATION

(71) Applicant: MEDIAIPLUS, INC., Seongnam-si (KR)

(72) Inventors: Yong Jang JO, Seongnam-si (KR); Ji Hee JUNG, Seongnam-si (KR)

(21) Appl. No.: 19/388,957

(22) Filed: Nov. 13, 2025

(30) Foreign Application Priority Data
Nov. 25, 2024 (KR) 10-2024-0169807

(51) Int. Cl. G16H 10/20; G06Q 30/018

(52) U.S. Cl. CPC G16H 10/20; G06Q 30/018

(57) ABSTRACT

Provided are an apparatus and a method for recommending a clinical trial contract research organization by analyzing a requirement included in a request for proposal (RFP). A clinical trial contract research organization matching apparatus according to an exemplary embodiment includes a converter which generates two types of variable vectors generated according to formats of requirements included in a request for proposal (RFP) for a clinical trial contract research organization received from a user, a recommender which recommends one or more clinical trial contract research organizations based on two types of variable vectors, and an output unit which outputs one or more recommended clinical trial contract research organizations according to a predetermined format.

### CROSS-REFERENCE TO RELATED APPLICATIONS

This application claims the priority of Korean Patent Application No. 10-2024-0169807 filed on Nov. 25, 2024, in the Korean Intellectual Property Office, the disclosure of which is incorporated herein by reference.

### BACKGROUND

### Field

The present disclosure relates to an apparatus and a method for recommending a clinical trial contract research organization by analyzing requirements included in a request for proposal (RFP).

### Description of the Related Art

In the related art, a sponsor has a difficulty in evaluating the capability of a clinical trial contract research organization due to lack of systematic and reliable channels for collecting key information, such as performance records of the clinical trial contract research organization, countries eligible for clinical trials, the number of clinical trials conducted by disease and drug type, and analytical equipment. For this reason, the sponsor tends to rely on the personal networks of a consultant specialized in matching clients and clinical trial contract research organization, which may result in contracts with clinical trial contract research organization being concluded at prices higher than the market rate.

In Korean Laid-Open Patent Publication No. 10-2017-0027576, an apparatus and method of researcher recommendation based on matching studying career is disclosed.

This invention was filed with support from the “2025 Global Startup Commercialization Support Program” funded by Gyeonggi Province and the Gyeonggi Business & Science Accelerator.

### SUMMARY

An object of the present disclosure is to provide an apparatus and a method for recommending a clinical trial contract research organization by analyzing requirements included in a request for proposal (RFP).

According to an aspect, a clinical trial contract research organization matching apparatus may include a converter which generates two types of variable vectors generated according to formats of requirements included in a request for proposal (RFP) for a clinical trial contract research organization received from a user; a recommender which recommends one or more clinical trial contract research organizations based on two types of variable vectors; and an output unit which outputs one or more recommended clinical trial contract research organizations according to a predetermined format.

The converter generates a binary variable vector for a binary-choice type requirement, among one or more requirements included in the request for proposal and may generate a continuous variable vector for other types of requirements.

The recommender may generate a mutual information matrix based on mutual information for each element of the binary variable vector.

The recommender may generate correlation matrix based on Spearman correlation for each element of the continuous variable vector.

The converter may generate the binary variable vector and the continuous variable vector for the organization data according to a predetermined rule, based on data about one or more clinical trial contract research organizations corresponding to the request for proposal.

The recommender may generate a binary variable matrix by calculating an outer product between a binary variable vector for the requirement and a binary variable vector for the organization data.

The recommender calculates an outer product between a continuous variable vector for the requirement and a continuous variable vector for organization data to generate a continuous variable matrix.

The recommender may generate a binary Hadamard product matrix by performing Hadamard product on a mutual information matrix and the binary variable matrix.

The recommender may generate the continuous Hadamard product matrix by performing Hadamard product on the correlation matrix and the continuous variable matrix.

The recommender may calculate the binary variable score and the continuous variable score by adding elements of the binary Hadamard product matrix and the continuous Hadamard product matrix.

The recommender may calculate an evaluation score for one or more clinical trial contract research organizations by calculating a weighted sum of each of the binary variable score and the continuous variable score.

The recommender sorts one or more clinical trial contract research organizations based on the evaluation score for one or more clinical trial contract research organizations and may recommend one or more clinical trial contract research organizations based on a ranking of the one or more sorted clinical trial contract research organizations and a work scope of the one or more clinical trial contract research organizations.

The output unit displays a user's location and one or more recommended clinical trial contract research organizations on a map and may determine a displaying color of the one or more recommended clinical trial contract research organizations based on the ranking of the clinical trial contract research organizations.

The clinical trial contract research organization matching apparatus may further include an estimator which generates estimate information for at least one of the one or more recommended clinical trial contract research organizations and determines estimate adequacy.

The clinical trial contract research organization matching apparatus further includes a contract unit which provides a contract error analysis and contract editing function for a clinical trial contract research organization.

According to an aspect, a clinical trial contract research organization matching method which is carried out on a computing device including one or more processors and a memory which stores one or more programs executed by the one or more processors, may include a converting step of generating two types of variable vectors generated according to formats of requirements included in a request for proposal (RFP) for a clinical trial contract research organization received from a user; a recommending step of recommending one or more clinical trial contract research organizations based on two types of variable vectors; and an output step of outputting one or more recommended clinical trial contract research organizations according to a predetermined format.

In the converting step, a binary variable vector for a binary-choice type requirement, among one or more requirements included in the request for proposal, is generated and a continuous variable vector for other types of requirements may be generated.

In the recommending step, a mutual information matrix may be generated based on mutual information for each element of the binary variable vector.

In the recommending step, a correlation matrix may be generated based on Spearman correlation for each element of the continuous variable vector.

In the converting step, the binary variable vector and the continuous variable vector for the organization data according to a predetermined rule may be generated based on data about one or more clinical trial contract research organizations corresponding to the request for proposal.

In the recommending step, a binary variable matrix may be generated by calculating an outer product between a binary variable vector for the requirement and a binary variable vector for the organization data.

In the recommending step, an outer product between a continuous variable vector for the requirement and a continuous variable vector for organization data are generated to generate a continuous variable matrix.

In the recommending step, a binary Hadamard product matrix may be generated by performing Hadamard product on a mutual information matrix and the binary variable matrix.

In the recommending step, the continuous Hadamard product matrix may be generated by calculating a Hadamard product on the correlation matrix and the continuous variable matrix.

In the recommending step, the binary variable score and the continuous variable score may be calculated by adding elements of the binary Hadamard product matrix and the continuous Hadamard product matrix.

In the recommending step, an evaluation score for one or more clinical trial contract research organizations may be calculated by calculating a weighted sum of each of the binary variable score and the continuous variable score.

In the recommending step, one or more clinical trial contract research organizations are sorted based on the evaluation score for one or more clinical trial contract research organizations and one or more clinical trial contract research organizations may be recommended based on a ranking of the one or more sorted clinical trial contract research organizations and a work scope of the one or more clinical trial contract research organizations.

In the output step, a user's location and one or more recommended clinical trial contract research organizations are displayed on a map and a displaying color of the one or more recommended clinical trial contract research organizations may be determined based on the ranking of the clinical trial contract research organizations.

The clinical trial contract research organization matching method may further include an estimating step of generating estimate information for at least one of the one or more recommended clinical trial contract research organizations and determining estimate adequacy.

The clinical trial contract research organization matching method may further include a contract step of providing a contract error analysis and contract editing function for a clinical trial contract research organization.

A clinical trial contract research organization is recommended by analyzing the request for proposal, thereby determining a clinical trial contract research organization having a capability desired by a user.

The effects of the present disclosure are not limited to the aforementioned effects, and various other effects are included in the present specification.

### BRIEF DESCRIPTION OF THE DRAWINGS

The above and other aspects, features and other advantages of the present disclosure will be more clearly understood from the following detailed description taken in conjunction with the accompanying drawings, in which:

FIG. 1 is a diagram of a clinical trial contract research organization matching apparatus according to an exemplary embodiment;

FIGS. 2, 3, 4, 5, 6, 7 and 8 are exemplary diagrams for explaining a clinical trial contract research organization matching method according to an exemplary embodiment; and

FIG. 9 is a flowchart illustrating a clinical trial contract research organization matching method according to an exemplary embodiment.

### DETAILED DESCRIPTION OF THE EMBODIMENT

Hereinafter, an exemplary embodiment of the present disclosure will be described in detail with reference to the accompanying drawings. In the description of the present disclosure, a detailed description of known configurations or functions incorporated herein will be omitted when it is determined that the detailed description may make the subject matter of the present disclosure unclear. Further, the terms to be described below are defined considering the functions in the present disclosure and may vary depending on the intention or usual practice of a user or operator. Accordingly, the terms need to be defined based on details throughout this specification.

Hereinafter, exemplary embodiments of a clinical trial contract research organization matching apparatus and a clinical trial contract research organization matching method will be described in detail with reference to the drawings.

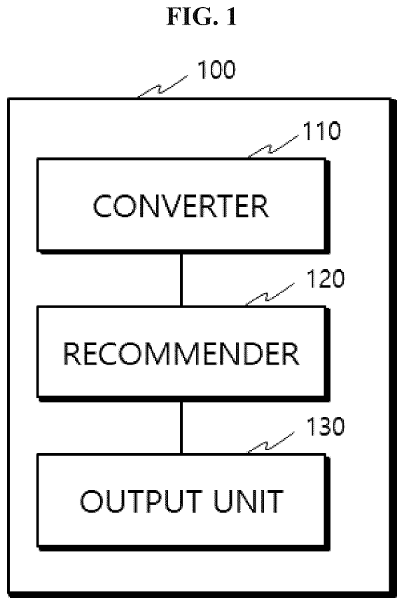

FIG. 1 is a diagram of a clinical trial contract research organization matching apparatus according to an exemplary embodiment.

According to an exemplary embodiment, a clinical trial contract research organization matching apparatus 100 may include a converter 110, a recommender 120, and an output unit 130.

According to an exemplary embodiment, the converter 110 may generate two types of variable vectors generated for each format of the requirements included in the request for proposal (RFP) for the clinical trial contract research organization received from a user.

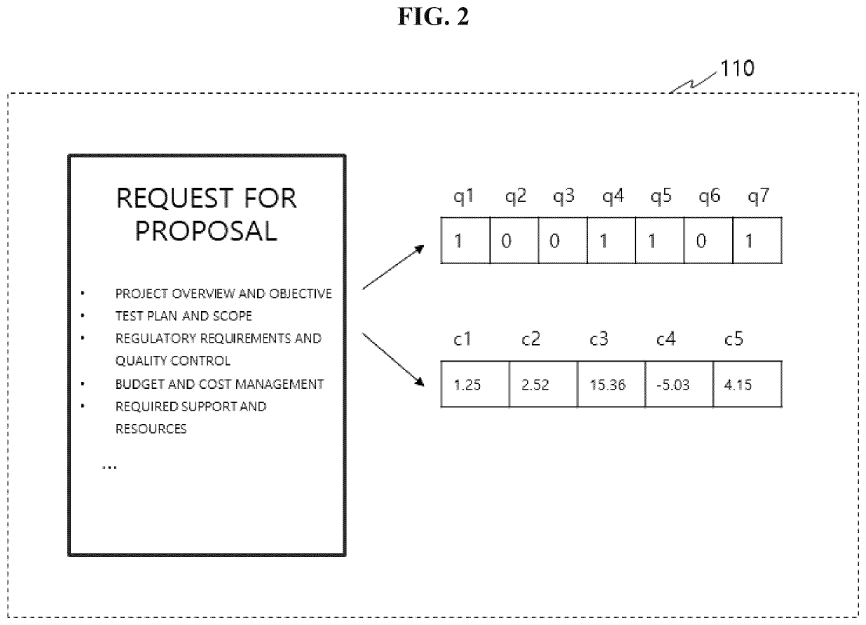

For example, the converter 110 generates a binary variable vector for a binary-choice type requirement, among one or more requirements included in the request for proposal and may generate a continuous variable vector for other types of requirements. Referring to FIG. 2, the converter 110 analyzes the received request for proposal to generate a binary variable vector for a binary-choice type requirement in which input data is classified into two values, such as “YES” or “NO, among one or more requirements. Further, a continuous variable vector for the other requirements may be generated. For example, the analyzer 110 analyzes the received request for proposal to generate the binary variable vector having a value of 0 or 1, for [q1, q2, . . . , q7] and generate the continuous variable vector having a real number value for [c1, c2, . . . , c5].

For example, the converter 110 may generate the binary variable vector and the continuous variable vector for the organization data according to a predetermined rule, based on data about one or more clinical trial contract research organizations corresponding to the request for proposal. For example, requests for proposal which previously matched the clinical trial contract research organizations according to the previously performed matching result may be stored in a database.

For example, one or more requests for proposal may match for each clinical trial contract research organization, such as request 1 for proposal for clinical trial contract research organization 1 and request 2 for proposal for clinical trial contract research organization 2. At this time, when a plurality of requests for proposal matches one clinical trial organization, a representative request for proposal may be generated according to a predetermined rule. For example, a latest request for proposal may be determined as the representative request for proposal or the plurality of requests for proposal is collected to determine a representative request for proposal.

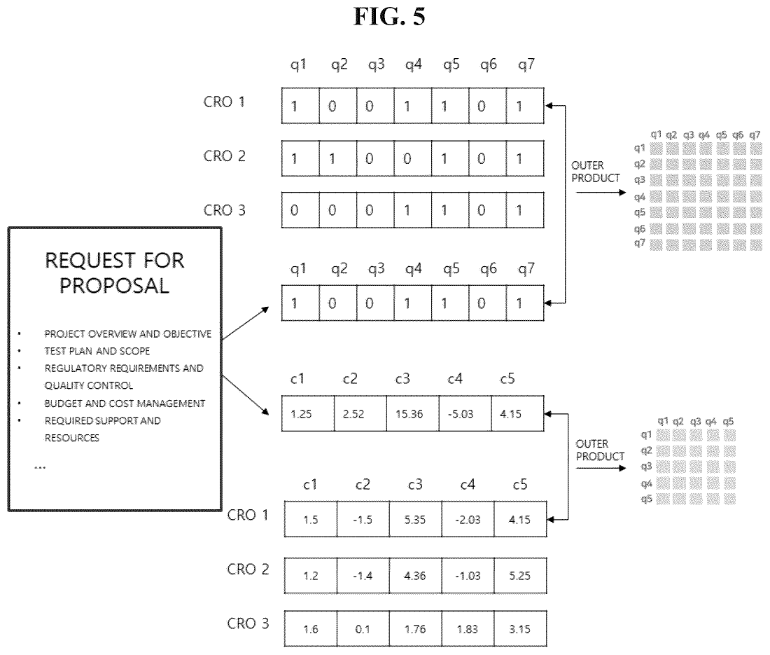

For example, the converter 110 may generate a binary variable vector and a continuous variable vector for the organization data, based on one or more requests for proposal which match each clinical trial contract research organization. For example, the matching unit 110 may generate a binary variable vector CRO1 and a continuous variable vector CRO1 for Clinical trial contract research organization 1 as illustrated in FIG. 5, according to the method illustrated in FIG. 2.

According to an exemplary embodiment, the recommender 120 may recommend one or more clinical trial contract research organizations based on two types of variable vectors. After receiving the request for proposal from the user, the recommender 120 recommends various clinical trial contract research organizations (CRO) based on the received request for proposal. During this process, the recommender 120 may use a predetermined matching algorithm. For example, the recommender 120 may calculate a predetermined score for recommending a clinical trial contract research organization using data which is processed as binary and continuous variables through the matching algorithm. Items utilized for the matching may include specific requirements for the clinical trial, expertise of the organization, past outputs, and other key indicators.

According to an example, the recommender 120 primarily selects one or more clinical trial contract research organizations based on the score which is calculated based on the request for proposal of the user and the characteristic of the clinical trial contract research organization. Next, the recommender 120 may derive a recommendation list from the selected organizations through more accurate filtering.

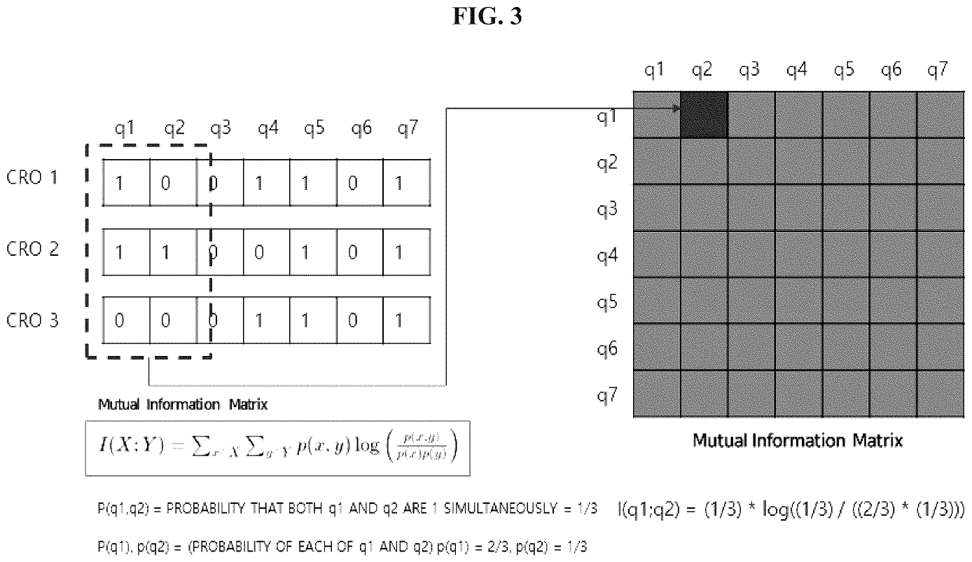

According to the exemplary embodiment, the recommender 120 may generate a mutual information matrix based on mutual information for each element of the binary variable vector. Referring to FIG. 3, the recommender 120 may use mutual information as a tool for measuring a correlation between elements of the binary variable vector which is provided as an input. The binary variable has a value of 0 or 1 and the mutual information is a measure indicating dependency between two variables. That is, information which is provided for one variable by another variable may be measured. For example, the mutual information I(X;Y) is defined by the following Equation.

$$I ( X ; Y ) {\sum }_{x ∈ X} {\sum }_{y ∈ Y} log ( \frac{p ( x , y )}{p ( x ) p ( y )} ) \qquad [ Equation 1 ]$$

Here, X and Y are two random variables and p(x, y) represents a probability (joint probability) that two variables simultaneously have specific values. Further, p(x) and p(y) represent individual probabilities of the variables X and Y, respectively.

The recommender 120 may calculate mutual information between two variables for each binary variable pair using the mutual information. If a total number of binary variables is n, the mutual information for all variable pairs is calculated to configure a mutual information matrix of n×n. Each element of the matrix represents mutual information between the corresponding variable pair. At this time, the mutual information matrix has the following features. First, mutual information always has zero or a positive value and the value of zero means that information is not shared between two variables. Second, mutual information is symmetrical. That is, I(X;Y)=I(Y;X) and this means that the extend to which information of X predicts Y is equal to the extend to which information of Y predicts X. Finally, the mutual information is normalized and relative information between two variables may be compared using the normalized mutual information.

According to the exemplary embodiment, the recommender 120 may generate correlation matrix based on Spearman correlation for each element of the continuous variable vector.

For example, the recommender 120 calculates the Spearman correlation between elements of the continuous variable vector to generate the correlation matrix. The Spearman correlation converts observed values of each variable into ranks and then may measure the relationship between two variables based on the ranking difference between the variable pair. For example, the correlation which uses the ranking difference dᵢ is represented by the following Equation.

$$ρ = 1 - \frac{6 \sum {d}_{i}^{2}}{n ( {n}^{2} - 1 )} \qquad [ Equation 2 ]$$

Here, dᵢ is a ranking difference of each observed value pair, n is the number of observed values, and r is a Spearman correlation. The recommender 120 determines the ranking difference by calculating the correlation for all variable pairs of the given continuous variable vector and measures a correlation level between two variables based on the ranking difference, and then may represent the result with the correlation matrix. Each element of the correlation matrix is filled with Spearman correlation between corresponding variable pairs and visualizes the relationship between the variables. For example, the magnitude of the correlation matrix is m×m and if there are m continuous variables, the correlation between the variables may be analyzed.

According to the exemplary embodiment, the recommender 120 calculates an outer product between a binary variable vector for the requirement and a binary variable vector for organization data to generate a binary variable matrix.

According to an example, the converter 110 analyzes requirements given in the request for proposal to generate a binary variable vector [q1, q2, . . . , q7] having a value of 0 or 1 and generates a binary variable vector such as [i1, i2, . . . , i7] from the organization data. Next, the recommender 120 calculates an outer product between the binary variable vector [q1, q2, . . . , q7] for the requirements and the binary variable vector [i1, i2, . . . , i7] as illustrated in FIG. 5 to generate a 7×7 binary variable matrix. At this time, the matrix may indicate matching information about how much the characteristics of the requirement and the organization match. Each element of the matrix is represented by 0 or 1 and shows a matching result for a combination of the requirement and the organization data.

According to the exemplary embodiment, the recommender 120 calculates an outer product between a continuous variable vector for the requirement and a continuous variable vector for organization data to generate a continuous variable matrix.

For example, the converter 110 generates the continuous variable vector [c1, c2, . . . , c5] represented by variables which may have continuous values for some items of the request for proposal and may generate the continuous variable vector, such as [ic1, ic2, . . . , ic5] from the organization data. Next, the recommender 120 may generate a 5×5 continuous variable vector by means of the outer product of the continuous variable vector [c1, c2, . . . , c5] for the requirement and the continuous variable vector [ic1, ic2, . . . , ic5] for the organization data. This matrix may represent how much the values of the continuous variable are similar and may express a matching degree as a numerical value. The recommender 120 may evaluate the compatibility between the organization and the request for proposal during the matching process using the binary variable matrix and the continuous variable matrix which are generated as described above.

According to the exemplary embodiment, the recommender 120 may generate a binary Hadamard product matrix by performing Hadamard product on the mutual information matrix and the binary variable matrix. Further, the recommender 120 may generate the continuous Hadamard product matrix by calculating a Hadamard product on the correlation matrix and the continuous variable matrix.

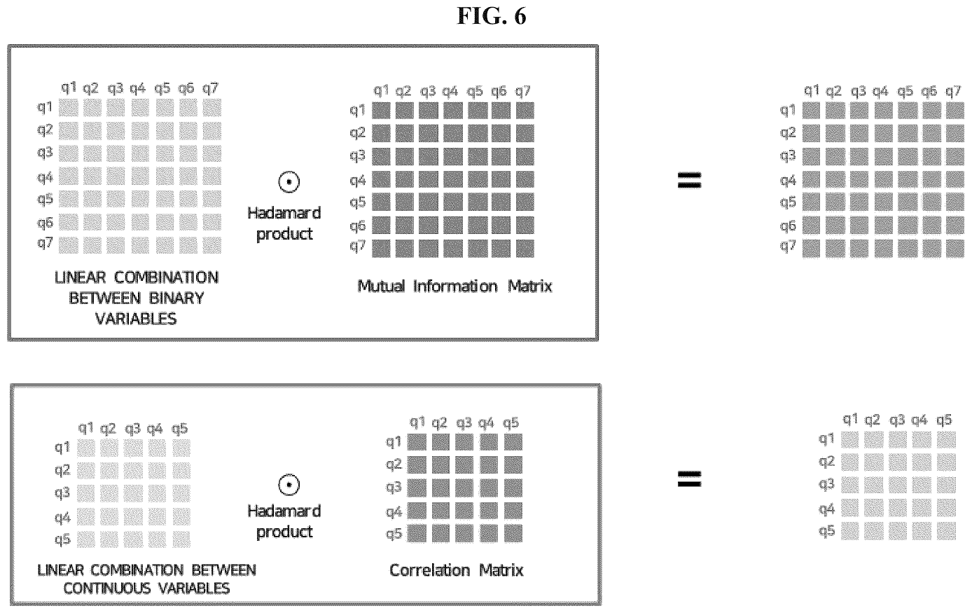

Referring to FIG. 6, the recommender 120 performs the outer product operation using request information which is newly input and CRO entrustment information in the database to generate a linear combination between the binary variables and a linear combination between continuous variables. Next, the recommender 120 may calculate the Hadamard product on the linear combination between the binary variables and the mutual information matrix and the linear combination between the continuous variables and the correlation matrix, respectively. By doing this, the mutual information or the correlation between the variables is calculated to evaluate the dependency between information. The recommender 120 may quantitatively evaluate the compatibility between the Hadamard product matrix generated and the request information and the CRO.

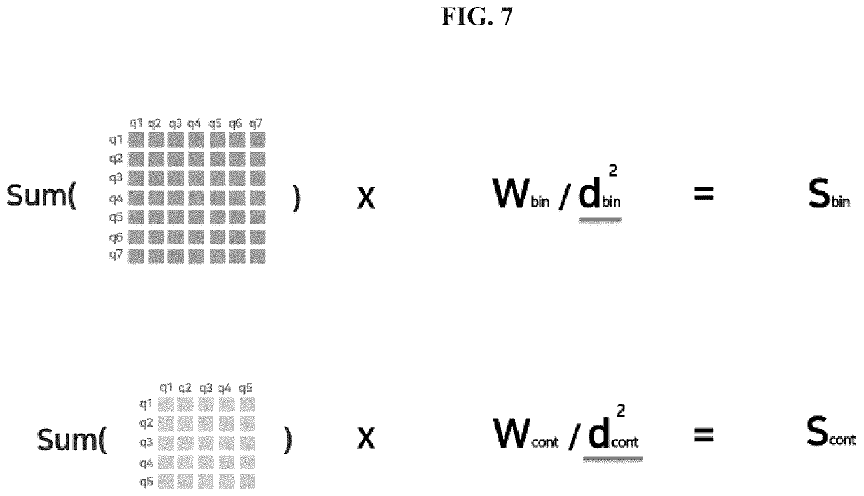

According to the exemplary embodiment, the recommender 120 adds elements of the binary Hadamard product matrix and the continuous Hadamard product matrix to calculate the binary variable score and the continuous variable score. Referring to FIG. 7, the recommender 120 adds all the elements of the binary Hadamard product matrix and the continuous Hadamard product matrix to calculate the binary variable score and the continuous variable score. During this process, a similarity score may be calculated by adding all the elements of each matrix, multiplying a weight W, and then dividing the result by a square d² of the dimension of the matrix. The scores which are independently calculated for the binary variable and the continuous variable represent the similarity between the binary variable and the continuous variable.

According to the exemplary embodiment, the recommender 120 may calculate an evaluation score for one or more clinical trial contract research organizations through a weight-sum of the binary variable score and the continuous variable score. For example, the recommender 120 adds all the elements of rows and columns of the binary variable matrix and the continuous variable matrix to calculate two variables. Next, the recommender 120 obtains a weighted sum of two variables. For example, a weight for the weighted sum may be determined based on magnitudes of the binary variable matrix and the continuous variable matrix. By doing this, the recommender 120 calculates a final evaluation score for each organization and may recommend an appropriate clinical trial contract research organization based on the final evaluation score.

According to the exemplary embodiment, the recommender 120 sorts one or more clinical trial contract research organizations based on the evaluation score for one or more clinical trial contract research organizations and may recommend one or more clinical trial contract research organizations based on a ranking of the one or more sorted clinical trial contract research organizations and a work scope of the one or more clinical trial contract research organizations.

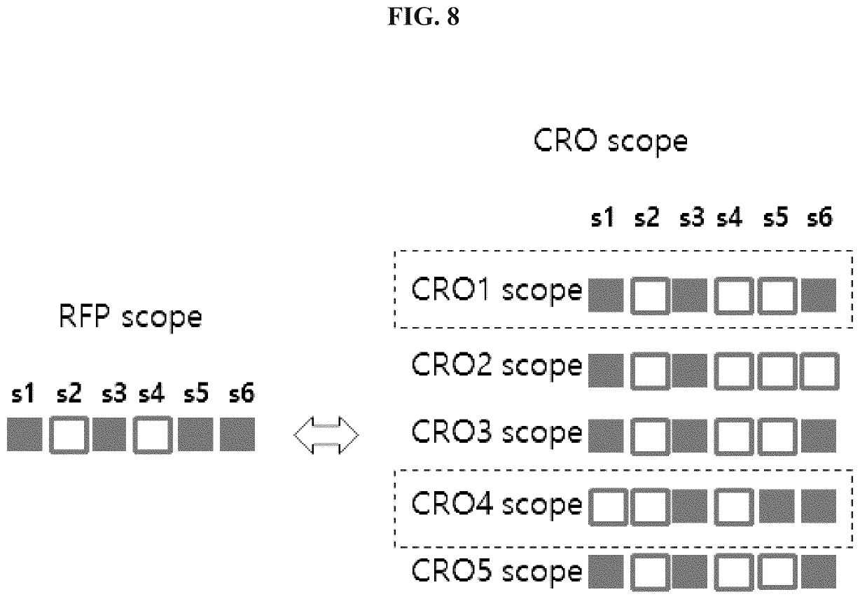

According to an example, the recommender 120 may sort the clinical trial contract research organizations based on the evaluation score for each clinical trial contract research organization. Referring to FIG. 8, the recommender 120 may sort the clinical trial contract research organizations, such as CRO1, CRO2, . . . , in descending order of the evaluation scores. Next, the recommender 120 determines how much a work scope (Scope) of a predetermined number of clinical trial contract research organizations having high evaluation scores and the requirements (Scope) match.

According to an example, the recommender 120 evaluates the work scopes s1, s2, and s3 of each clinical trial contract research organization and determines how much the work scopes match the requirement of the user. For example, as illustrated in FIG. 8, the clinical trial contract research organizations may be sorted in the order of CRO1, 2, 3, 4, and 5 and the work scope (Scope) of each CRO is reviewed again in the sorted order to determine an organization which satisfies the user's requirements s1, s3, s5, and s6. For example, the clinical trial contract research organizations corresponding to CRO1 and CRO4 may be determined as organizations which satisfy the user's requirements scopes 1, 3, 5, and 6. The recommender 120 may finally recommend CRO1 and CRO4 to the user based on the sorted ranking and the work scope (Scope) of each organization.

According to the exemplary embodiment, the output unit 130 may output one or more recommended clinical trial contract research organizations according to a predetermined format. For example, the output unit 130 displays the user's location and one or more recommended clinical trial contract research organizations on a map and may determine a displaying color of one or more recommended clinical trial contract research organizations based on the ranking of the clinical trial contract research organizations.

According to an example, the output unit 130 may visualize the list of the recommended clinical trial contract research organizations with respect to the user's location, by utilizing the map. During this process, the location of each clinical trial contract research organization is visualized with colors or intensity to easily identify the ranking or the compatibility of each organization. If the user selects the corresponding clinical trial contract research organization, the output unit 130 may output additional information for each sub-item to allow the user to intuitively identify the suitability of the clinical trial contract research organization.

According to the exemplary embodiment, the clinical trial contract research organization matching apparatus 100 may further include an estimator (not illustrated) which generates estimate information about at least one of one or more recommended clinical trial contract research organizations and determines an estimate adequacy.

According to an example, the estimator may manage the processes of generating, correcting, submitting, and reviewing the estimate between the clinical trial contract research organization and the user. According to the example, the estimator provides an estimate template and the request for proposal of the client to the matched clinical trial contract research organizations to allow the clinical trial contract research organization to create the estimate based on the provided information. The estimator may provide a function of identifying an error and a corrected part of the estimate and a memo function. By doing this, the clinical trial contract research organization may easily find and correct a part which needs to be corrected.

According to the exemplary embodiment, if an error is found as a result of determining the estimate adequacy, the estimator transmits error information to a clinical trial contract research organization which transmits the estimate having the error and if no error is found as the result of determining the estimate adequacy, transmits the estimate received from the clinical trial contract research organization to the user.

After completing the estimate preparation, the estimator may analyze a data format, a scope, content consistency, and missing portions of the estimate submitted by the clinical trial contract research organization. If an error is found as an analysis result, the estimator transmits the request for correction to the clinical trial contract research organization and the portion which needs to be corrected may be identified and annotated with a note. The clinical trial contract research organization corrects the error within a specified uploading period and then submits the estimate and during this process, a notification function and automatic access link may be provided.

If it is determined that there is no error as the analysis result, the estimator transmits the estimate to the user and provides the user with a notification function and a function to see the estimate via the automatic access link. A download validity period is set for the estimate and a security measure, such as a password may be included to grant an access right for reviewing the estimate.

According to the exemplary embodiment, the estimator generates an estimated cost based on a previously stored estimate for one or more recommended clinical trial contract research organizations to generate estimate information and compares the estimated cost received from the one or more recommended clinical trial contract research organizations and an expected estimated cost to determine the estimate adequacy. According to the example, the estimator visualizes the cost comparison based on the past and current data to determine the adequacy of the cost proposed by the clinical trial contract research organization. By doing this, a level of the estimated cost proposed by the clinical trial contract research organization as compared with an average of the past and current costs may be visually presented to the user.

According to the exemplary embodiment, the clinical trial contract research organization apparatus 100 may further include a contract unit (not illustrated) which provides a contract error analysis and contract editing function for the clinical trial contract research organization.

According to an example, the contract unit may perform the contract error analysis for the clinical trial contract research organization using an artificial neural network trained to review at least one of the data format, the content consistency, and the content missing.

According to an example, the contract unit automatically generates a contract room and may manage all the contract related processes in this space. The contract unit may automatically generate and provide a customized contract template based on basic information and estimate contents of the user and the clinical trial contract research organization.

According to the example, the contract unit analyzes the missing part, the content consistency, and formality errors of the contract to support the user to easily correct them. Further, the contract unit may notify all relevant users about the corresponding situation whenever the contract is created, corrected, and submitted and generates and provides automatic links to allow the users to conveniently access the contract. For example, the contract unit 120 may perform the contract error analysis using an artificial neural network trained to review important elements of the contract, such as the data format, the content consistency, and the content missing. The neural network is trained based on various contract datasets and may detect an error which may occur in the contract related to the clinical trial contract research organization thereby.

The contract unit visually identifies a part of the contract which needs to be corrected to provide the user with the identified part and the user may discuss and correct each item through the memorandum. Further, the contract unit provides a function of adding or deleting a specific clause of the contract under the agreement of the user and the clinical trial contract research organization. The contract unit tracks all records of the correction and the download of the contract and monitors all the records to ensure the integrity and the security of the contract. All records are stored to prevent the forgery, and any changes of the contract may be clearly managed.

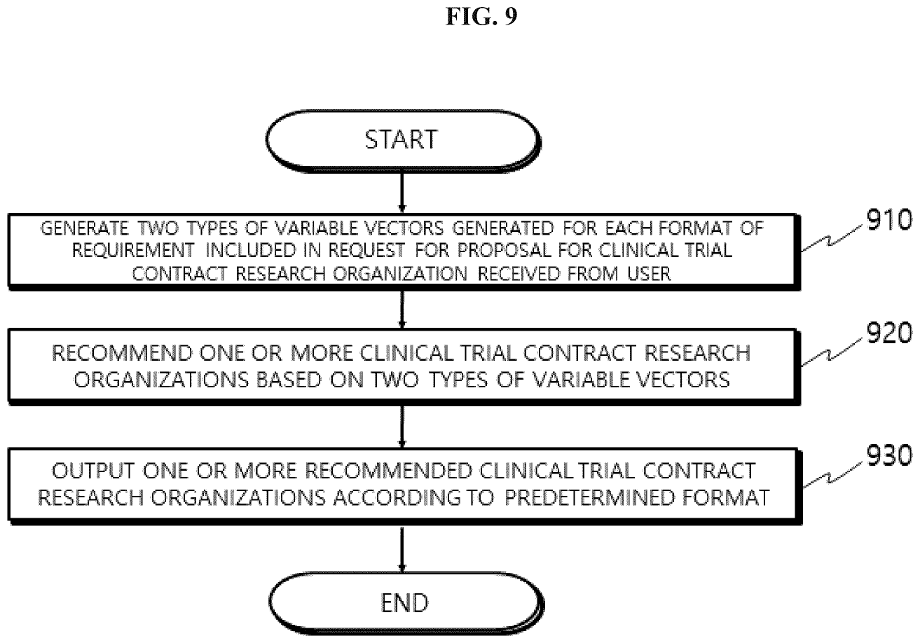

FIG. 9 is a flowchart illustrating a clinical trial contract research organization matching method according to an exemplary embodiment.

According to an exemplary embodiment, the clinical trial contract research organization matching apparatus may be a computing device including one or more processors and a memory which stores one or more programs executed by one or more processors.

According to an exemplary embodiment, the clinical trial contract research organization matching apparatus may generate two types of variable vectors generated according to formats of the requirements included in requests for proposal (RFP) for the clinical trial contract research organizations received from the user (910) and may recommend one or more clinical trial contract research organizations based on two types of variable vectors (920). Next, the clinical trial contract research organization matching apparatus may output one or more recommended clinical trial contract research organizations according to a predetermined format (930).

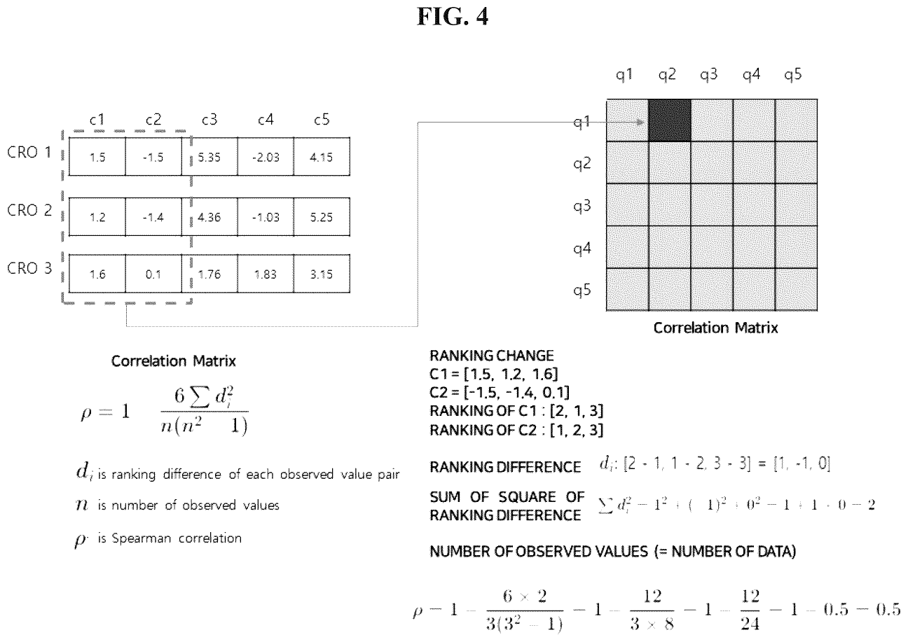

Among the embodiments of FIG. 9, embodiments that overlap the contents described with reference to FIGS. 1 to 8 are omitted.

An aspect of the present disclosure may also be implemented as computer-readable codes written on a computer-readable recording medium. Codes and code segments which implement the program may be easily deducted by a computer programmer in the art. The computer readable recording medium may include all kinds of recording devices in which data, which are capable of being read by a computer system, are stored. Examples of the computer-readable recording media may include ROM, RAM, CD-ROM, magnetic tape, floppy disk, optical disk and the like. Further, the computer readable recording medium is distributed in computer systems connected through a network to be written and executed with a computer readable code in a distributed manner.

For now, the present disclosure has been described with reference to the exemplary embodiments. It is understood to those skilled in the art that the present disclosure may be implemented as a modified form without departing from an essential characteristic of the present disclosure. Accordingly, the scope of the present disclosure is not limited to the above-described embodiment, but should be construed to include various embodiments within the scope equivalent to the description of the claims.

What is claimed is:

1. A clinical trial contract research organization matching apparatus, comprising:
a converter which generates two types of variable vectors generated according to formats of requirements included in a request for proposal (RFP) for a clinical trial contract research organization received from a user;
a recommender which recommends one or more clinical trial contract research organizations based on two types of variable vectors; and
an output unit which outputs one or more recommended clinical trial contract research organizations according to a predetermined format.

2. The clinical trial contract research organization matching apparatus according to claim 1, wherein the converter generates a binary variable vector for a binary-choice type requirement, among one or more requirements included in the request for proposal and generates a continuous variable vector for the other requirements.

3. The clinical trial contract research organization matching apparatus according to claim 2, wherein the recommender generates a mutual information matrix based on mutual information for each element of the binary variable vector.

4. The clinical trial contract research organization matching apparatus according to claim 3, wherein the recommender generates a correlation matrix based on Spearman correlation for each element of the continuous variable vector.

5. The clinical trial contract research organization matching apparatus according to claim 4, wherein the converter generates a binary variable vector and a continuous variable vector for organization data based on data about one or more clinical trial contract research organizations corresponding to the request for proposal according to a predetermined rule.

6. The clinical trial contract research organization matching apparatus according to claim 5, wherein the recommender generates a binary variable matrix by calculating an outer product between a binary variable vector for the requirement and a binary variable vector for the organization data.

7. The clinical trial contract research organization matching apparatus according to claim 6, wherein the recommender generates a continuous variable matrix by calculating an outer product between a continuous variable vector for the requirement and a continuous variable vector for the organization data.

8. The clinical trial contract research organization matching apparatus according to claim 7, wherein the recommender generates a binary Hadamard product matrix by performing Hadamard product on a mutual information matrix and the binary variable matrix.

9. The clinical trial contract research organization matching apparatus according to claim 8, wherein the recommender generates a continuous Hadamard product matrix by performing Hadamard product on the mutual matrix and the continuous variable matrix.

10. The clinical trial contract research organization matching apparatus according to claim 9, wherein the recommender calculates a binary variable score and a continuous variable score by adding elements of each of the binary Hadamard product matrix and the continuous Hadamard product matrix.

11. The clinical trial contract research organization matching apparatus according to claim 10, wherein the recommender calculates an evaluation score for one or more clinical trial contract research organizations by calculating a weighted sum of each of the binary variable score and the continuous variable score.

12. The clinical trial contract research organization matching apparatus according to claim 11, wherein the recommender sorts one or more clinical trial contract research organizations based on the evaluation scores of the one or more clinical trial contract research organizations and recommends one or more clinical trial contract research organizations based on a ranking of the one or more sorted clinical trial contract research organizations and a work scope of the one or more clinical trial contract research organizations.

13. The clinical trial contract research organization matching apparatus according to claim 12, wherein the output unit displays a user's location and one or more recommended clinical trial contract research organizations on a map and determines a displaying color of the one or more recommended clinical trial contract research organizations based on the ranking of the clinical trial contract research organizations.

14. The clinical trial contract research organization matching apparatus according to claim 1, further comprising:
an estimator which generates estimate information for at least one of the one or more recommended clinical trial contract research organizations and determines estimate adequacy.

15. The clinical trial contract research organization matching apparatus according to claim 14, further comprising:
a contract unit which provides a contract error analysis and contract editing function for a clinical trial contract research organization.

16. A clinical trial contract research organization matching method which is carried out on a computing device including one or more processors and a memory which stores one or more programs executed by the one or more processors, the method comprising:
a converting step of generating two types of variable vectors generated according to formats of requirements included in a request for proposal (RFP) for a clinical trial contract research organization received from a user;
a recommending step of recommending one or more clinical trial contract research organizations based on two types of variable vectors; and
an output step of outputting one or more recommended clinical trial contract research organizations according to a predetermined format.

17. The clinical trial contract research organization matching method according to claim 16, wherein in the converting step, a binary variable vector for a binary choice type requirement, among one or more requirements included in the request for proposal, is generated and a continuous variable vector for the other requirements is generated.

18. The clinical trial contract research organization matching method according to claim 17, wherein in the recommending step, a mutual information matrix is generated based on mutual information for each element of the binary variable vector.

19. The clinical trial contract research organization matching method according to claim 18, wherein in the recommending step, a correlation matrix is generated based on Spearman correlation for each element of the continuous variable vector.

20. The clinical trial contract research organization matching method according to claim 19, wherein in the converting step, a binary variable vector and a continuous variable vector for organization data are generated based on data about one or more clinical trial contract research organizations corresponding to the request for proposal according to a predetermined rule.

21. The clinical trial contract research organization matching method according to claim 20, wherein in the recommending step, a binary variable matrix is generated by calculating an outer product between the binary variable vector for the requirement and the binary variable vector for the organization data.

22. The clinical trial contract research organization matching method according to claim 21, wherein in the recommending step, a continuous variable matrix is generated by calculating an outer product between a continuous variable vector for the requirement and a continuous variable vector for the organization data.

23. The clinical trial contract research organization matching method according to claim 22, wherein in the recommending step, a binary Hadamard product matrix is generated by performing Hadamard product on a mutual information matrix and the binary variable matrix.

24. The clinical trial contract research organization matching method according to claim 23, wherein in the recommending step, a continuous Hadamard product matrix is generated by performing Hadamard product on the mutual matrix and the continuous variable matrix.

25. The clinical trial contract research organization matching method according to claim 24, wherein in the recommending step, a binary variable score and a continuous variable score are calculated by adding elements of each of the binary Hadamard product matrix and the continuous Hadamard product matrix.

26. The clinical trial contract research organization matching method according to claim 25, wherein in the recommending step, an evaluation score for one or more clinical trial contract research organizations is calculated by calculating a weighted sum of each of the binary variable score and the continuous variable score.

27. The clinical trial contract research organization matching method according to claim 26, wherein in the recommending step, one or more clinical trial contract research organizations are sorted based on the evaluation scores of the one or more clinical trial contract research organizations and one or more clinical trial contract research organizations are recommended based on a ranking of the one or more sorted clinical trial contract research organizations and a work scope of the one or more clinical trial contract research organizations.

28. The clinical trial contract research organization matching method according to claim 27, wherein in the output step, a user's location and one or more recommended clinical trial contract research organizations are displayed on a map and a displaying color of the one or more recommended clinical trial contract research organizations is determined based on the ranking of the clinical trial contract research organizations.

29. The clinical trial contract research organization matching method according to claim 16, further comprising:
an estimating step of generating estimate information for at least one of the one or more recommended clinical trial contract research organizations and determining estimate adequacy.

30. The clinical trial contract research organization matching method according to claim 29, further comprising:
a contract step of providing a contract error analysis and contract editing function for a clinical trial contract research organization.
